(19) United States
(12) Patent Application Publication
(10) Pub. No.: US 2017/0189243 A1
(43) Pub. Date: Jul. 6, 2017

(54) DIAPER

(71) Applicant: SJ CIRCLE SDN BHD, Kulim (MY)

(72) Inventor: Sew Jin YEOH, Butterworth (MY)

(21) Appl. No.: 15/394,283

(22) Filed: Dec. 29, 2016

(30) Foreign Application Priority Data
Dec. 31, 2015 (MY) PI2015704853

(51) Int. Cl. A61F 13/49

(52) U.S. Cl. CPC A61F 13/4902; A61F 13/49011; A61F 2013/49031; A61F 2013/49034; A61F 2013/49041

(57) ABSTRACT

The present invention generally relates a diaper (1) for infant or adult, comprising, an anterior portion (AP) capable of encapsulating a human anterior pelvic region, a posterior portion (PP) capable of encapsulating a human posterior pelvic region, and a center portion (CP) capable of encapsulating a human pelvic region, couples the anterior portion (AP) and posterior portion (PP) middle region (MR), perpendicularly length-wise.

### 1. TECHNICAL FIELD OF THE INVENTION

The present invention generally relates a diaper for infant and adult, comprising, an anterior portion capable of encapsulating a human anterior pelvic region, a posterior portion capable of encapsulating a human posterior pelvic region, and a center portion capable of encapsulating a human pelvic region, couples the anterior portion and posterior portion middle region, perpendicularly length-wise.

### 2. BACKGROUND OF THE INVENTION

Diaper is well known to provide household convenience in disposing infant and adult waste in a wearable form. Due to increasing human population growth, thus, diaper demand has been increasing dramatically. As a result, diaper manufacturers are facing a daunting challenge to reduce material usage while not compromising the diaper quality. Thus, the present invention teaches a diaper that uses lesser material as compare to the conventional diapers.

### 3. SUMMARY OF THE INVENTION

Accordingly, it is the primary aim of the present invention to provide material-saving diaper.

It is yet an object of the present invention to provide a singular group of sinusoidal waveform-like elastic bands disposed on the posterior portion of the diaper.

According to the preferred embodiment of the present invention the following is provided,

A diaper (1) for infant and adult, comprising:

an anterior portion (AP) capable of encapsulating a human anterior pelvic region;

a posterior portion (PP) capable of encapsulating a human posterior pelvic region; and

a center portion (CP) capable of encapsulating a human pelvic region, couples the anterior portion (AP) and posterior portion (PP) middle region (MR), perpendicularly length-wise;

characterized in that

the anterior portion (AP) comprising at least one layer of non-woven portion (AP1, AP2) with a group of elastic bands in straight continuous line arranged length-wise (L) embedded the portion (AP1, AP2) therein; and

the posterior portion (PP) comprising at least one layer of non-woven portion (PP1) with at least one elastic band in continuous curvilinear (12-C) and rectilinear (12-R) arranged length-wise (L), and at least one layer of non-woven portion (PP2) with at least one elastic band in continuous line arranged in mirrored and length-wise (L) embedded the portion (PP1, PP2) therein.

### 4. BRIEF DESCRIPTION OF THE DRAWINGS

Other aspect of the present invention and their advantages will be discerned after studying the Detailed Description in conjunction with the accompanying drawings in which:

FIG. 1 shows an exemplary front view of the present invention.

FIG. 2 shows an exemplary front view of an S-elastic band applied on the diaper.

FIG. 3-A shows a portion of S-elastic band superimposed over the side of a Gluteus maximus muscle group.

FIG. 3-B shows a portion of S-elastic band superimposed over the Gluteus maximus muscle group.

FIG. 4-A shows an exemplary front view of preparing S-elastic band on non-woven sheet.

FIG. 4-B shows an exemplary front view of preparing a group of straight elastic bands on non-woven sheet.

FIG. 4-C shows an exemplary front view of aligning non-woven sheet in FIG. 4-A and non-woven sheet in FIG. 4-B.

FIG. 4-D shows an exemplary front view of merging both non-woven sheets comprising straight elastic bands and S-elastic bands.

FIG. 4-E shows a front view of merged non-woven sheet is cut in the cutting line.

FIG. 4-F shows a front view of merged non-woven sheet of PP (posterior portion) where the excess region outside of the S-elastic bands is removed.

FIG. 4-G shows a front view of merged non-woven sheet of PP (posterior portion) and AP (anterior portion) after the excess region outside of the S-elastic bands are removed.

FIG. 4-H shows a front view of a plurality of center portions are placed/coupled with the PP (posterior portion) and AP (anterior portion), then outer edges of PP and AP are aligned and abut each other.

FIG. 4-I shows an enlarged top view of merged non-woven sheet posterior portion (PP) and anterior portion (AP) are joined with a center portion (CP) comprising absorbent materials, to form a diaper, prior to a (i) adhesive means, heat sealing, or ultrasonic sealing on the width axis of PP and AP edges, and also prior to (ii) a cutting process along the cutting line on the width axis.

FIG. 5-A shows a side view of the present invention.

FIG. 5-B shows a top view of the present invention.

### 5. DETAILED DESCRIPTION OF THE DRAWINGS

In the following detailed description, numerous specific details are set forth in order to provide a thorough understanding of the invention. However, it will be understood by the person having ordinary skill in the art that the invention may be practised without these specific details. In other instances, well known methods, procedures and/or components have not been described in detail so as not to obscure the invention.

The invention will be more clearly understood from the following description of the embodiments thereof, given by way of example only with reference to the accompanying drawings, which are not drawn to scale.

Throughout this disclosure of diaper manufacturing, diaper non-woven sheets are under a tensile force as they are being conveyed by a plurality of rollers. The tensile force is adjustable by manipulating speed between rollers; increasing the roller speed momentarily will increase the non-woven sheet tensile force; decreasing the roller speed momentarily shall reduce the non-woven sheet tensile force, conversely. Application of elastic bands, application of adhesive, aligning the non-woven sheets, and application of heat sealing are carried out while the non-woven sheets are under the tensile force.

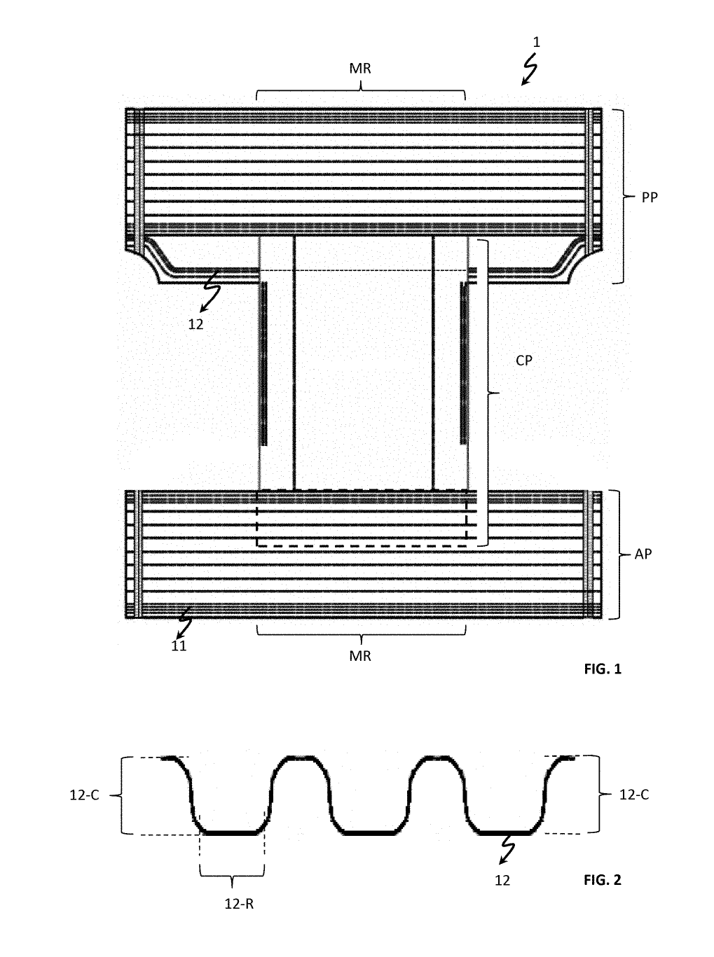

Referring to FIG. 1, there is shown an exemplary diaper for infant and adult. The diaper (1) comprising an anterior portion (AP) capable of encapsulating a human anterior pelvic region, a posterior portion (PP) capable of encapsulating a human posterior pelvic region, and a center portion (CP) capable of encapsulating a human pelvic region, couples the anterior portion (AP) and posterior portion (PP) middle region (MR), perpendicularly length-wise.

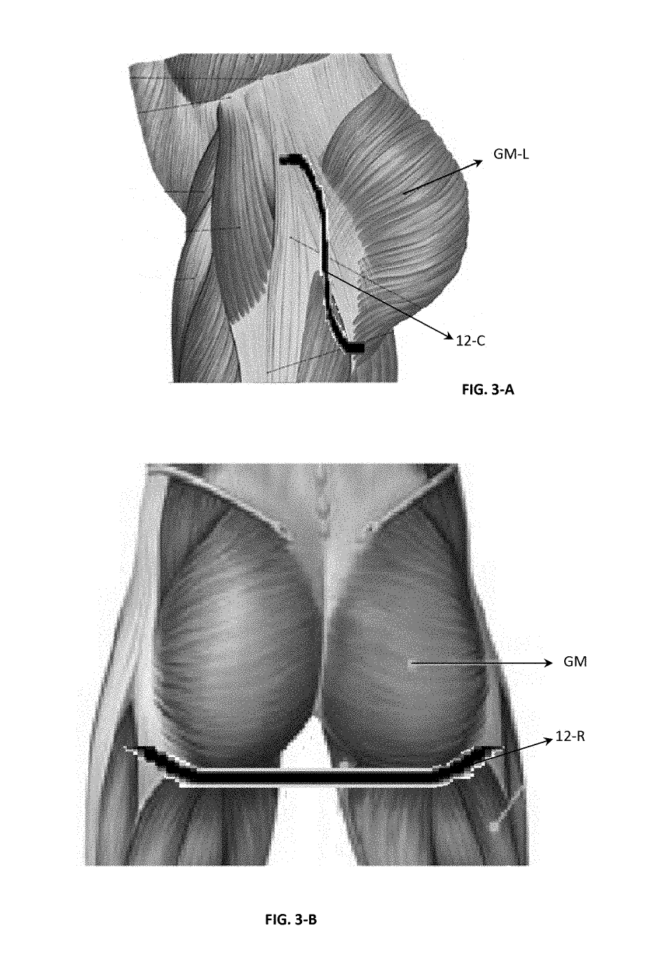

Referring to FIG. 2, there is shown an exemplary elastic band (12) akin to a sinusoidal waveform, in a continuous curvilinear (12-C) and rectilinear (12-R) is disposed and arranged in length-wise (L), on the diaper (1), preferably on the posterior portion (PP). Referring to FIGS. 3-A and 3-B, there are shown the continuous curvilinear (12-C) elastic band on the diaper (1) is disposed and worn on lateral sides of a muscle group gluteus maximus (GM-L). The continuous rectilinear (12-R) elastic band on the diaper (1) is disposed and worn on a muscle group gluteus maximus (GM).

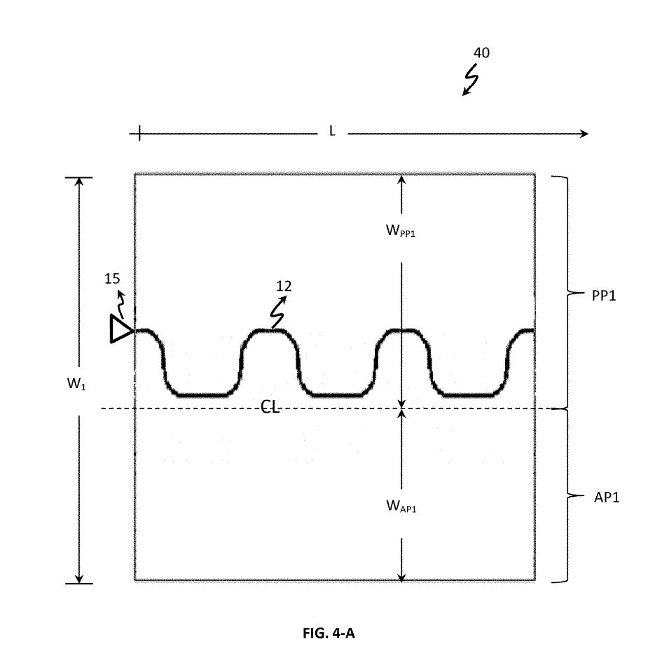

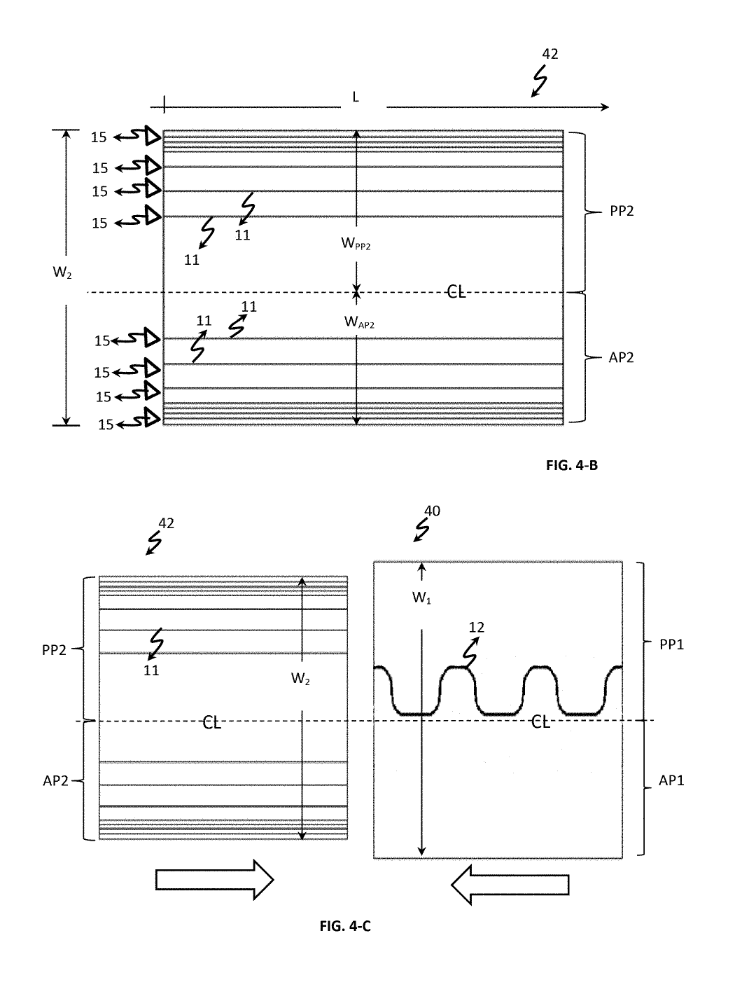

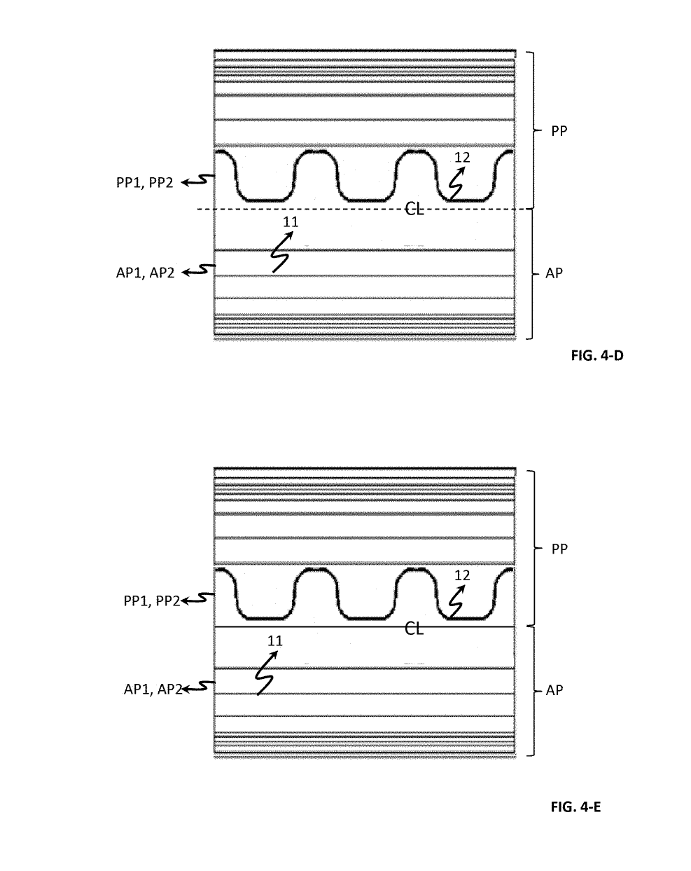

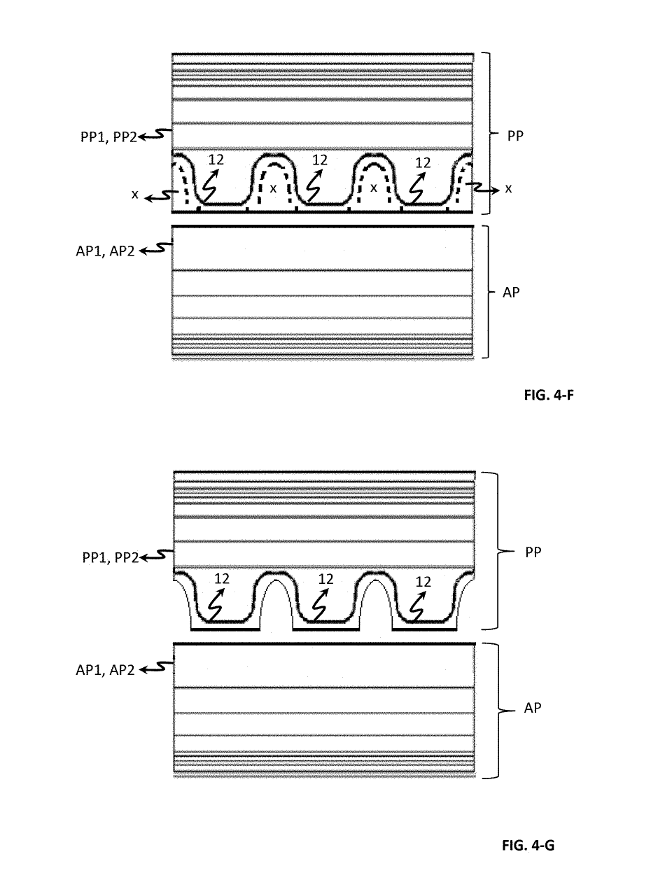

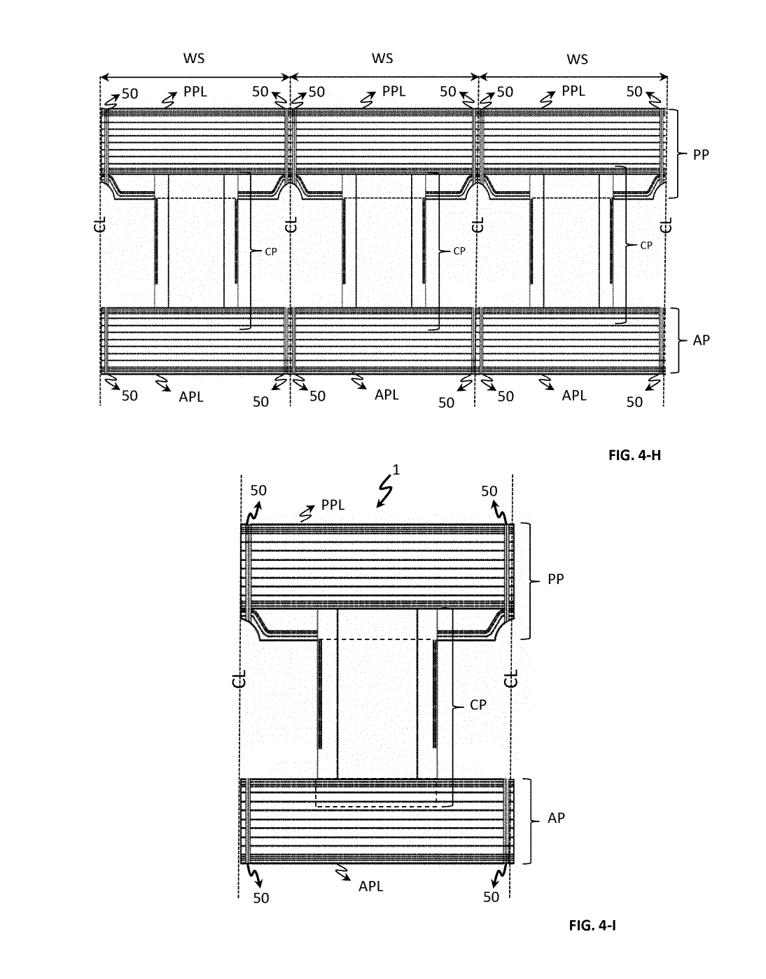

Referring to FIG. 4-A, there is shown an exemplary to preparing sinusoidal elastic band (12) on a non-woven sheet (40). The non-woven sheet (40) has a fixed width (W₁) and variable length (L). The sheet (40) has a cutting line (CL) demarcated by dotted line, along the sheet (40) length axis. The sheet (40) is divided into two portions by the cutting line (CL), namely non-woven posterior portion 1 (hereinafter referred to as PP1) and non-woven anterior portion 1 (hereinafter referred to as AP1), preferably, the width PP1 (W_{PP1}) is wider than the width AP1 (W_{AP1}). The non-woven portion (PP1) has at least one elastic band in continuous curvilinear (12-C) and rectilinear (12-R) arranged length-wise (L), disposed near the cutting line (CL) of the non-woven sheet (40). Both portions PP1 and AP1 are derived from the continuous non-woven sheet (40).

The non-woven portion PP1 surface is then applied at least one continuous curvilinear (12-C) and rectilinear (12-R) elastic band thereon by at least one elastic and adhesive applicator means (15). The elastic and adhesive applicator means (15) applies adhesive surrounding the area consisting the band (12). The portion AP1 does not contain any elastic band.

Referring to FIG. 4-B, there is shown shows an exemplary front view of preparing a group of straight elastic bands (11) by a plurality of elastic and adhesive applicator means (15) on a non-woven sheet (42). The sheet (42) is divided into two portions by the cutting line (CL), namely non-woven posterior portion 2 (hereinafter referred to as PP2) and non-woven anterior portion 2 (hereinafter referred to as AP2), preferably, the width PP2 (W_{PP2}) is wider than the width AP2 (W_{AP2}). The elastic and adhesive applicator means (15) capable of applying both adhesive and elastic band simultaneously, the adhesive is applied surrounding the elastic band (11). The non-woven portion (AP2) and the non-woven portion (PP2) have at least one elastic band in straight continuous line arranged in mirrored and length-wise (L), disposed away from the cutting line (CL) of the non-woven sheet (42). Again, the non-woven sheet (42) has a fixed width (W₂) and variable length (L). Yet again, it is preferable that PP2 has a bigger width (W^{PP2}) than AP2 width (W_{AP2}). However, the non-woven sheet (42) has less width (W₂) as compare to the non-woven sheet (40) width (W₁).

Referring now to FIG. 4-C, there is shown an exemplary front view of aligning non-woven sheet (40) and non-woven sheet (42) by the cutting line (CL). Prior to this preparation, adhesive is applied on both non-woven sheets (40, 42) to allow a lamination or merging, the merging direction is indicated by block arrows. Then, the non-woven sheets (40, 42) are aligned at the cutting line (CL) and then both sheets (40, 42) are laminated.

Referring now to FIGS. 4-D and 4-E, there are shown an exemplary front view of non-woven sheet (40) comprising at least one layer of posterior portion 1 (PP1) with sinusoidal elastic band (12) and at least one layer of anterior portion 1 (AP1) has no elastic bands. Therefore, the anterior portion (AP) comprising at least one layer of non-woven sheet (AP1, AP2) with a group of elastic bands in continuous line arranged length-wise (L) embedded the layer (AP1, AP2) therein.

Meanwhile, the non-woven sheet (42) comprising at least one layer of posterior portion 2 (PP2) and at least one layer of anterior portion 2 (AP2) comprising straight elastic bands (11). Therefore, the posterior portion (PP) comprising at least one layer of non-woven portion (PP1) with at least one elastic band in continuous curvilinear (12-C) and rectilinear (12-R) arranged length-wise (L), and at least layer of non-woven portion (PP2) with at least one elastic band in continuous line arranged length-wise (L) embedded the layer of portions (PP1, PP2) therein.

In FIG. 4-E, the merged non-woven sheets (40, 42) are separated by a cutting means along the cutting line (CL), thereby forming a non-woven posterior portion (PP) comprising PP1 and PP2, and a non-woven anterior portion (AP) comprising AP1 and AP2.

Referring now to FIG. 4-F, there is shown a front view of merged non-woven sheet of PP (posterior portion) where the excess regions (x) outside of the S-elastic bands (12) is removed. FIG. 4-G shows a front view of merged non-woven sheet of PP (posterior portion) and AP (anterior portion) after the excess regions (x) outside of the S-elastic bands (12) are removed.

Referring now to FIG. 4-H, there is shown a front view of a plurality of center portions (CP) are placed/coupled perpendicularly length-wise, with the PP (posterior portion) and AP (anterior portion) middle region by adhesive means.

Outermost edges of PP (PPL) and AP (APL) along the length (L) axis, are aligned and abut each other. Then an adhesive means, heat sealing, ultrasonic sealing (50), or a combination thereof, is applied on and coupled both PP and AP on the width (W) axis according to waist size (WS). A cutting means separates the diapers according to waist size (WS) along the cutting line (CL) width axis.

Referring now to FIG. 4-I, there is shown an enlarged top view of merged non-woven sheet posterior portion (PP) and anterior portion (AP) are joined with a center portion (CP) comprising absorbent materials, to form a diaper (1), prior to a (i) adhesive sealing, heat sealing, or ultrasonic sealing (50) on the width (W) axis of PP and AP, and also prior to (ii) a cutting process along the cutting line (CL) on the width (W) axis.

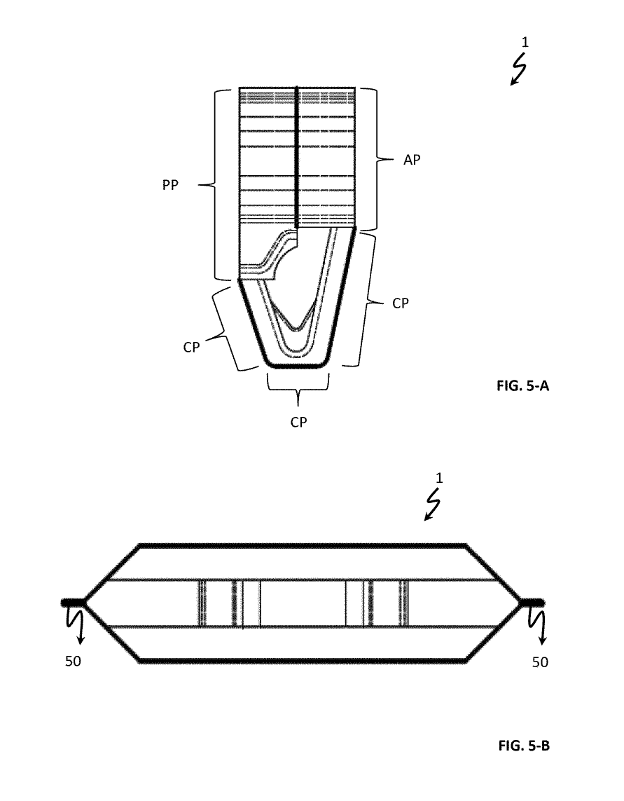

Referring now to FIG. 5-A, there is shown a side view of merged non-woven sheet posterior portion (PP) and anterior portion (AP) are joined with a center portion (CP) comprising absorbent materials, to form a diaper (1). The center portion (CP) capable of encapsulating a human pelvic region, couples the anterior portion (AP) and posterior portion (PP) middle region (MR), perpendicularly length-wise. Referring now to FIG. 5-B, the non-woven sheets (PP, AP) are coupled by adhesive means, heat sealing, ultrasonic seal, or combination thereof, along the width axis (W), to enable the diaper (1) wearable.

While the present invention has been shown and described herein in what are considered to be the preferred embodiments thereof, illustrating the results and advantages over the prior art obtained through the present invention, the invention is not limited to those specific embodiments. Thus, the forms of the invention shown and described herein are to be taken as illustrative only and other embodiments may be selected without departing from the scope of the present invention, as set forth in the claims appended hereto.

1. A diaper for infant and adult, comprising:
an anterior portion capable of encapsulating a human anterior pelvic region;
a posterior portion capable of encapsulating a human posterior pelvic region; and
a center portion capable of encapsulating a human pelvic region, couples the anterior portion and posterior portion middle region, perpendicularly length-wise;
wherein
the anterior portion comprising at least one layer of non-woven portion with a group of elastic bands in straight continuous line arranged length-wise embedded the portion therein; and
the posterior portion comprising at least one layer of non-woven portion with at least one elastic band in continuous curvilinear and rectilinear arranged length-wise, and at least one layer of non-woven portion with at least one elastic band in continuous line arranged in mirrored and length-wise embedded the portion therein.

2. The diaper as claimed in claim 1 wherein the continuous curvilinear elastic band on the diaper is disposed and worn on lateral sides of a muscle group gluteus maximus.

3. The diaper as claimed in claim 1 wherein the continuous rectilinear elastic band on the diaper is disposed and worn on a muscle group gluteus maximus.

4. The diaper as claimed in claim 1 wherein the non-woven portion and the non-woven portion are derived from a continuous non-woven sheet.

5. The diaper as claimed in claim 4 wherein the continuous non-woven sheet comprising the non-woven portion width is wider than the non-woven portion width.

6. The diaper as claimed in claim 1 wherein the non-woven portion and the non-woven portion are derived from a continuous non-woven sheet.

7. The diaper as claimed in claim 6 wherein the continuous non-woven sheet comprising the non-woven portion width is wider than the non-woven portion width.

8. The diaper as claimed in claims 4 wherein the non-woven sheet is wider than the non-woven sheet.

9. The diaper as claimed in claim 4 wherein the non-woven portion has no elastic band.

10. The diaper as claimed in claim 4 wherein the non-woven portion has at least one elastic band in continuous curvilinear and rectilinear arranged length-wise, disposed near the cutting line of the non-woven sheet.

11. The diaper as claimed in claim 6 wherein the non-woven portion and the non-woven portion have at least one elastic band in continuous line arranged length-wise, disposed away from the cutting line of the non-woven sheet.

12. The diaper as claimed in claim 1 wherein the non-woven sheets are coupled by adhesive means, heat sealing, ultrasonic sealing, or combination thereof, along the width axis.

13. The diaper as claimed in claim 1 wherein the continuous curvilinear and rectilinear akin to a sinusoidal waveform.
